(19) United States
(12) Patent Application Publication
(10) Pub. No.: US 2026/0167655 A1
(43) Pub. Date: Jun. 18, 2026

(54) Enantiomeric Compounds and Methods for Treating Invasive Tumors

(71) Applicant: Donald Barbeau, Savannah, GA (US)

(72) Inventor: Donald Barbeau, Savannah, GA (US)

(21) Appl. No.: 18/831,365

(22) Filed: Dec. 13, 2024

(51) Int. Cl. C07F 9/6558; A61K 31/5415; A61K 31/675; A61K 45/06; C07D 417/06

(52) U.S. Cl. CPC C07F 9/65583; A61K 31/5415; A61K 31/675; A61K 45/06; C07D 417/06

(57) ABSTRACT

The present invention relates to purified enantiomers of compounds and pharmaceutical compositions for treating a subject having a cancer in need of therapy thereof comprising administering to the subject a compound in an amount effective in inhibiting tumor growth, said compound having the formula:

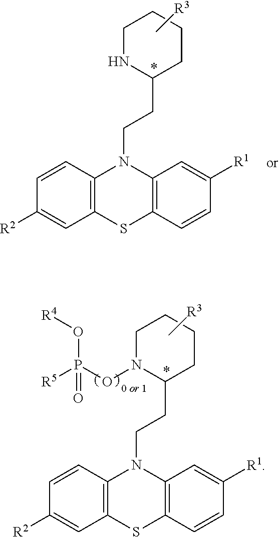

### BACKGROUND OF THE INVENTION

Cancer is a complex and heterogenous neoplastic disease. When normal cells become cancerous, they acquire several distinct biochemical properties that enable localized tumor growth in a variety of organs through uncontrolled cell division. When tumor cells become invasive and disseminate to distant metastatic sites in the body, they acquire additional biochemical properties that are distinct from both normal cells and cancer cells found in primary tumors. Metastasis relies on the epithelial-to-mesenchymal transition (EMT), signals from the tumor microenvironment and induced in response to therapy. Metastatic cancer remains incurable, and most cancer-related deaths are a result of complications from recurrent or metastatic disease. The inability to achieve sustained control of tumor cell proliferation and metastasis defines the resistance to current therapeutics, which target the biochemical properties of epithelial cancer cells in primary and metastatically established tumors. Current cancer therapies have little, if any, impact on patient survival.

One group of structurally related compounds (10-alkyl substituted phenothiazines) has routinely demonstrated control of tumor cell proliferation and metastasis that have evaded currently used therapeutics. These compounds are heteroaromatic lipophilic compounds having a planar aromatic ring structure with a cationic center disposed adjacent the ring nitrogen. The antipsychotic thioridazine, a 10-alkyl substituted phenothiazine compound, has demonstrated effectiveness as an anticancer agent effective in controlling tumors that have become resistant to other therapies. Thioridazine interact with high affinity to the β1 integrin that is upregulated on the cell surface of metastatically-transformed cells. The interaction of thioridazine with this integrin β1-subunit disrupts the association with its extracellular ligands, reduces intracellular signaling, and inhibits migration.

The affinity of phenothiazines toward 31 integrin varies according to their chemical structure. Thioridazine has the strongest binding (IC₅₀=3.2 μM), followed by perphenazine (IC₅₀=6.2 μM), methiothepin (IC₅₀=7.2 μM), trifluoperazine (IC₅₀=11.7 μM), and mexitene (IC₅₀=13.02 μM). Unfortunately, 10-alkyl substituted phenothiazines also have a high affinity toward the dopamine D2 receptor: fluphenazine (Kᵢ=1.4 nM); trifluoperazine (Kᵢ=1.1 nM); perphenazine (Kᵢ=0.5 nM); chlorpromazine (Kᵢ=3.4 nM); and thioridazine (Kᵢ=9.8 nM) (Byland et al. The Journal of Pharmacology and Experimental Therapeutics 112: (1981) 81-86). Studies have shown that (+)-thioridazine and (−)-thioridazine are partially selective for dopamine D2 and dopamine D1 receptors respectively. (Svendson C N et al. Neuropharmacology 27 (1988) 1117-1134; Jortani S A et al. Forensic Science International 64 (1994) 165-170).

The high affinity of these phenothiazines toward the dopamine receptor have been shown to cause 1) dose-limiting dopaminergic side effects due to their strong affinity for dopamine D2 receptors and 2) dose-limiting cardiotoxicity caused by excessive prolongation of the QT interval, which is a direct result of its strong hERG binding. Alternatives to thioridazine have been developed to overcome these limitations. One such alternative is described in U.S. Pat. No. 9,198,916 which is specifically incorporated herein by reference. The compound described in Example 1 of U.S. Pat. No. 9,198,916 for example has demonstrated highly potent in vitro cytotoxic activity against drug-resistant tumors including intractable metastatic melanoma tumors carrying the BRAF mutation, the locally invasive estrogen-negative breast tumors found in inflammatory breast cancer, and the highly invasive estrogen-negative breast tumors found in triple-negative breast cancer.

Most studies on the phenothiazines for the treatment of cancer have been conducted with the racemic mixture of these compounds, that is the (+)-phenothiazine together with the (−)-phenothiazine enantiomers. Other studies though have used the individual enantiomers to study their effectiveness as antibacterial agents and anticancer agents. These studies demonstrate that the racemic mixture of (+)-phenothiazine together with the (−)-phenothiazine showed no significant differences in their efficacy compared with either of the individual (+)-phenothiazine and (−)-phenothiazine isomers (Antonsen S et al. SynOpen 4, (2020) 12-16; Spengler G et al. Anticancer Research 36 (2016): 5701-5706; Christensen J B et al. PLUS One 8 (3): e57493; Csonka A et al. In Vitro 27 (2013): 815-820). Csonka et al, and Spengler et al. additionally concluded that the separate use of one thioridazine enantiomer versus the other does not appear to provide any advantage over the racemic mixture for cancer therapy. In studies with another phenothiazine, promethazine, for the prevention of bone loss, the (+)-promethazine isomer demonstrated greater activity than either the (−)-promethazine isomer or the racemic mixture (U.S. Pat. No. 8,637,503). Jensen A S (PH.D Dissertation from the Medical Informatics Group, Department of Health Science and Technology, Aalborg University, Denmark 2014; Jensen A S et al. European Journal of Pharmacology (2015) January 15:747:7-12) found that both the (+)-thioridazine and the racemate cause greater prolongation of the ventricular action potential duration (APD) than (−)-thioridazine. Prolonged APD is one determinant of cardiotoxicity; however, because thioridazine causes several effects on ion channels Jensen reported that his results do not provide conclusive evidence that (−)-thioridazine is less likely to cause ventricular arrhythmia than the (+)-thioridazine or the racemate. Nevertheless, there are no studies on 10-alkyl substituted phenothiazine compounds such as those described in U.S. Pat. No. 9,198,916 that describe or suggest a preference in isomer selectivity (including the (+)-phenothiazine, the (−)-phenothiazine and the phenothiazine racemates) in relation to anticancer activity and cardiotoxicity.

### OBJECTS OF THE INVENTION

It is an object of the present invention to provide enantiomeric compounds useful for treating cancer patients that advantageously control therapy-resistant tumor cells and have low risk of cardiotoxicity.

It is a further object of the present invention to provide enantiomeric compounds useful for treating cancer patients that advantageously control therapy-resistant tumor cells, have low risk of causing ventricular arrhythmias, reduced dopaminergic side effects, reduced hERG binding and reduced QTc interval prolongation.

It is also an object of the present invention to provide a method for treating a subject in need of cancer therapy comprising administering to the subject an enantiomeric compound in an amount effective in inhibiting tumor growth, proliferation, differentiation, apoptosis, motility, or autophagy in patients.

It is a further object of the present invention to provide enantiomeric compounds useful for treating cancer patients in combination with immunotherapies.

It is a further object of the present invention to provide enantiomeric compounds useful for treating cancer patients in combination with iradiotherapy.

### BRIEF SUMMARY OF THE INVENTION

The present invention relates to purified enantiomers of compounds and pharmaceutical compositions for treating a subject having a cancer in need of therapy thereof comprising administering to the subject a compound in an amount effective in inhibiting tumor growth, said compound having the formula:

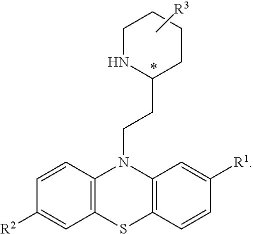

where R¹ is hydrogen, halogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, pharmaceutically acceptable salt or a controlled release carrier. The compounds of the invention advantageously control therapy-resistant tumor cells and have low cardiotoxicity risk.

### DETAILED DESCRIPTION OF THE INVENTION

In accordance with the present invention, provided are enantiomerically pure compounds having the formula:

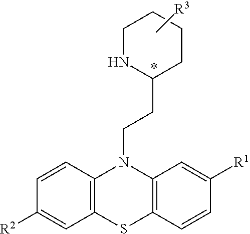

where R¹ is hydrogen, halogen, trifluoromethyl or an alkylthio group, R² is hydrogen, alkoxy or a branched or a straight chain acyclic alkyl group containing from one to about five carbon atoms, and R⁵ is hydrogen, lower alkyl or lower alkoxy, and a pharmaceutically acceptable carrier, pharmaceutically acceptable salt or a controlled release carrier.

In accordance with the present invention, R¹ is hydrogen, halogen, trifluoromethyl, sulfhydryl or an alkylthio group. In a preferred embodiment of the present invention, R¹ is trifluoromethyl or an alkylthio group. In accordance with the present invention, the alkylthio group is a sulfone wherein the sulfonyl group is connected to an alkyl having from 1 to about three carbon atoms and to a ring carbon. In a preferred embodiment of the present invention, the alkylthio is thiomethyl having the formula: —S(CH₃).

In accordance with the present invention, lower alkyls are those alkyls containing a branched or straight chain acyclic alkyl group containing from one to five carbon atoms and include methyl, ethyl, propyl, isopropyl, butyl, isobutyl and pentyl. In accordance with a preferred embodiment of the present invention, lower alkyls are those containing from 1 to about three carbon atoms. In accordance with the present invention, lower alkoxy groups include methoxy, ethoxy, propoxy such as n-propoxy, butoxy such as n-butoxy and t-butoxy, and pentoxy such as n-pentoxy. In accordance with the present invention, halogens are preferably fluorine and chlorine.

In accordance with the present invention, R² is hydrogen, alkoxy or lower alkyl including but not limited to methyl, ethyl, propyl or butyl. In accordance with a preferred embodiment of the present invention, R² is hydrogen, alkoxy or a branched or a straight chain acyclic alkyl group containing from one to about three carbon atoms, including but not limited to methyl, ethyl, and propyl. In one embodiment of the present invention, R² is preferably hydrogen. In another embodiment of the present invention R² is an alkoxy or a branched or a straight chain acyclic alkyl group containing from one to about five carbon atoms. In accordance with a preferred embodiment of the present invention, R² is propyl.

In accordance with the present invention, R³ is hydrogen, a lower alkyl or a lower alkoxy. In accordance with one embodiment of this invention, R³ is methyl or ethyl. In a more preferred embodiment of the present invention, R³ is hydrogen.

In accordance with a preferred embodiment of the present invention, provided are enantiomeric compounds having the formula:

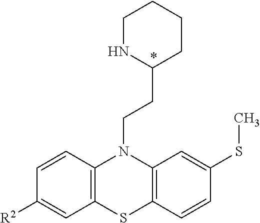

In accordance with the present invention purified enantiomers are those that are not a racemate or are non-superimposable mirror images of each other with a chiral center. In accordance with this invention, the optical rotation for the starting material, [2R)-piperidin-2-yl] acetic acid, for the R isomer was [α]25D=+0.1 (c=0.1, MeOH). Likewise, in accordance with the present invention, the optical rotation for the starting material, [(2S)-piperidin-2-yl] acetic acid, for the S isomer was [α]25D=+0.1 (c=0.1, MeOH).

The compounds of the present invention advantageously control therapy-resistant solid and hematological tumor cells and have low cardiotoxicity risk. In accordance with the present invention, “control” of tumors includes inhibiting tumor growth, proliferation, differentiation, apoptosis, motility, or autophagy and the like in cancer patients. Hematological tumors include leukemias. Solid cancers in which these tumors are found include, inter alia, bladder cancer, breast cancer, colorectal cancer, head and neck cancer, gastric cancer, esophageal cancer, ovarian cancer, cervical cancer uterine cancer, endometrial cancer, liver cancer, lung cancer, mesotheliomas, melanomas, pancreatic cancer, prostate cancer, renal cancer, thyroid cancer, gliomas including glioblastomas and diffuse intrinsic pontine gliomas, sarcomas and neuroblastomas.

The compounds of the present invention can be administered as monotherapy or in combination with radiotherapy or immunotherapy. Immunotherapy, in accordance with the present invention, includes administering T-cells, NK killer cells or CART cells to the subject. Natural killer (NK) cells are cytotoxic lymphocytes of the innate immune system that are capable of killing virally infected and/or cancerous cells. Natural killer (NK) cells are potent innate immune system effector lymphocytes armed with multiple mechanisms for killing cancer cells. Several strategies are being employed to increase their number and improve their ability to overcome cancer resistance and the immunosuppressive tumor microenvironment. These include the use of cytokines and synthetic compounds to bolster propagation and killing capacity, targeting immune-function checkpoints, addition of chimeric antigen receptors (CARs) to provide cancer specificity and genetic ablation of inhibitory molecules. In accordance with the present invention, immunotherapy also includes the inhibition of PD-L1. PD-L1 inhibitors are checkpoint inhibitor anticancer drugs that block the activity of PD-1 and PDL1 immune checkpoint proteins present on the surface of cells. Examples of PD-L1 inhibitors include durvalumab, atezolizumab and avelumab. Both PD-1 and PD-L1 inhibitors have been shown to be helpful in treating many different types of cancer.

The compounds of the present invention can be administered orally or by parenteral administration. In accordance with one embodiment of the present invention, the compounds are administered intravenously. In this embodiment, the presently claimed compounds provide an unexpected advantage over similar compounds in the prior art, particularly thioridazine. Thioridazine's cardiotoxicity in patients is believed to be caused by the excessive prolongation of QT interval (Hartigan-Go et al. Clinical Pharmacology & Therapeutics 60:543-553 (1996), and a direct result of its high hERG liability. Surprisingly and unexpectedly, the compounds of the present invention have an advantage over previously used phenothiazine compounds like thioridazine because they have a low hERG liability.

The compounds of the present invention can be administered to cancer patients having therapy-resistant tumors including, but not limited to drug-resistant tumors, metastatic tumors and subpopulations of cancer stem cells. The compounds of the present invention can be administered alone or in combination with other cancer therapies. Compounds of the present invention can be administered to a patient receiving one or more chemotherapeutic or targeted therapies, where the compound is administered before, during or after the chemotherapeutic or targeted therapy. Illustrative therapies that can be combined with the compounds of the present invention include chemotherapeutic drugs, targeted drugs, epigenetic drugs and the like.

In accordance with another embodiment of the present invention, the compounds of the present invention are administered orally. In this embodiment, the presently claimed compounds provide an additional unexpected advantage over similar compounds in the prior art, particularly thioridazine. Orally administered phenothiazines like thioridazine, for example, have demonstrated a propensity toward oxidation by cytochrome P450 2D6 enzymes to 7-hydroxy quinoneimine metabolites that have their oxidized (hydroxyl) group in the para-position to the ring nitrogen in the phenothiazine structure. Without being held to a particular theory or mechanism, we believe that the highly reactive quinoneimine toxicophores that are produced when phenothiazines-based drugs are administered orally are responsible in part for the proarrhythmic activity that has been reported to cause severe life-threatening arrhythmias (torsade de pointes) and sudden death. Compounds in which the highly reactive quinoneimine toxicophores are not produced provide a significant advantage over phenothiazines such as thioridazine.

The preparation and use of compounds and prodrugs in accordance with the present invention will be readily apparent to those skilled in the art and the well-known and well-documented procedures for substituted phenothiazine-based drugs in the scientific and patent literature. The following United States patents provide illustrations on the synthesis of the phenothiazine analogs, and are hereby incorporated by reference in their entirety, together with the patents cited therein: U.S. Pat. Nos. 2,905,590; 3,310,553; 4,107,430; 4,042,695; 3,951,961; 6,407,231; 5,503,759; 3,305,547 and 8,088,918.

Synthesis and preparation of enantiomeric compounds in accordance with the present invention can be prepared by those of ordinary skill in the art without undue experimentation as taught in U.S. Pat. No. 6,020,506, which is specifically incorporated herein by reference for the purpose of describing and enabling the preparation of compositions comprising enantiomerically pure compounds. Methods of purification will be well known to those of ordinary skill and may include, e.g., dissolution of the mixture in a solvent and recrystallization. Column chromatography may be used to resolve a phenothiazine racemate into its two enantiomers. Racemates may also be separated using chromatographic separation, such as gas chromatography (GC) or high-performance liquid chromatography (HPLC), such as used in the resolution of promethazine, ethopropazine, trimeprazine and trimipramine enantiomers (Ponder G W, Stewart J T. J Pharm Biomed Anal. 1995 August; 13 (9): 1161-6.) Capillary electrophoresis (CE) may also be employed (Wang et al., 2001). These compounds can also be easily prepared by the synthetic scheme or that described by Daniel et al. Pol. J. Pharmacol. 49 (6): 439-452 (1997); Daniel et al. Exp. Toxicol. Pathol. 51 (4-5): 309-314 (1999); Daniel et al. British Journal of Pharmacology 131:287-295 (2000). Starting materials for compounds where R² is other than hydrogen, for example, include lower alkyls such as 3-methyl-10H-phenothiazine (CAS 3939-47-7), 3-ethyl-10H-phenothiazine (CAS 54027-87-1), 3-propyl-10H-phenothiazine (CAS 92-33-1), 3-butyl-10H-phenothiazine (CID 70288115), 3-propan-2yl-10H-phenothiazine (CID 70290282), 3-(1,1 dimethylethyl)-10H-phenothiazine (CAS 7678-79-7) and alkoxy such as 3-methoxy-10H-phenothiazine (CAS 1771-19-3).

Compounds of the present invention can be prepared as prodrugs that avoid biotransformation by cytochrome P450 2D6 to quinoneimine metabolites. The preparation of these prodrugs is described in U.S. Pat. No. 8,088,918 which is hereby incorporated by reference. In accordance with another preferred embodiment of the present invention, compounds are provided having the formula:

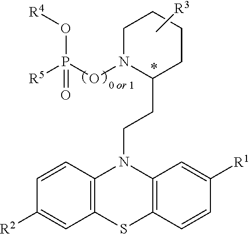

where R¹ is hydrogen, halogen, trifluoromethyl or an alkylthio group, R² is hydrogen, alkoxy or a branched or a straight chain acyclic alkyl group containing from about one to about five carbon atoms, and R³ is hydrogen, lower alkyl or lower alkoxy, and a pharmaceutically acceptable carrier, salt or a controlled release carrier.

Determining the dose and the amounts of compounds effective in treating a subject having a disease and in need of therapy, controlling or reducing tumor growth or reducing tumorigenicity in accordance with the present invention will be readily apparent to those skilled in the art. In accordance with one aspect of the present invention, a method for treating a subject having cancer comprises administering to the subject a compound in an amount effective in modulating in vivo tumor growth or tumorigenicity.

The compounds of the present invention can be administered in admixture with a suitable pharmaceutical excipient, diluent or carrier selected with regard to the intended route of administration and standard pharmaceutical practice. The term “carrier” refers to diluents, excipients and the like for use in preparing admixtures of a pharmaceutical composition. For example, the compounds of the present invention can be administered orally in the form of tablets, capsules, multi-particulates, elixirs, solutions or suspensions, which may contain flavoring or coloring agents, either for immediate-, delayed-, modified-, sustained-, pulsed- or controlled-release applications. Suitable formulations of the compounds of the present invention may be in coated or uncoated form, as desired. Prodrugs in accordance with the present invention may be pH-labile and require delayed-release formulations to protect the prodrug from hydrolysis in the stomach. Preferably these delayed-release formulations contain enteric coatings. Pharmaceutically acceptable carriers include but are not limited to sterile water, saline, buffered saline, dextrose solution, preferably such physiologically compatible buffers as Hank's or Ringer's solution, physiological saline, a mixture consisting of saline and glucose, and heparinized sodium-citrate-citric acid-dextrose solution and the like. As used herein, the term “pharmaceutically acceptable” means approved by a regulatory agency of the Federal or a state government or listed in the U.S. Pharmacopeia or other generally recognized pharmacopeia for use in animals, and more particularly in humans.

Unless otherwise specifically identified or claimed for preferred embodiments, the following general definitions are used in accordance with the present invention. In accordance with the present invention, the term “to target” or “to targeted” refers to the recognition of a target and delivery of a drug to that target; however, no internalization of the drug is inferred. In accordance with the present invention, the term “selectively target” refers to selective preference of one cell type over another. In accordance with the present invention, the term “modulate” refers to a change in the parameter measured, such that modulate can mean either an increase or decrease.

The present invention has been described in detail using specific examples to illustrate the preferred embodiments of the invention; however, it will be obvious to those skilled in the art that various modifications thereto can be made without departing from the spirit and scope thereof.

### EXAMPLES OF THE INVENTION

### Example 1:2-(methylsulfanyl)-10-{2-[(2R)-piperidin-2-yl]ethyl}-10Hphenothiazine dihydrochloride

2-(methylsulfanyl)-10-{2-[(2R)-piperidin-2-yl]ethyl}-10Hphenothiazine dihydrochloride was prepared using [(2R)-piperidin-2-yl]acetic acid as the purified enantiomer starting material. The optical rotation of the starting material was [α]25D=+0.1 (c=0.1, MeOH). 2-(methylsulfanyl)-10-{2-[(2R)-piperidin-2-yl] ethyl}-10Hphenothiazine dihydrochloride was produced in 95% yield by ¹H NMR analysis and 95.2% by LCMS analysis and appeared as a green crystalline powder. The elemental analysis for 2-(methylsulfanyl)-10-{2-[(2R)-piperidin-2-yl] ethyl}-10Hphenothiazine dihydrochloride is C20H26Cl2N2S2, the formula weight is 429.4698, and the moisture content using the “Karl Fisher” method is 1.40% (by weight).

### Example 2:2-(methylsulfanyl)-10-{2-[(2S)-piperidin-2-yl] ethyl}-10H-phenothiazine dihydrochloride

2-(methylsulfanyl)-10-[2-[(2S)-piperidin-2-yl]ethyl]-10H-phenothiazine dihydrochloride was prepared using [(2S)-piperidin-2-yl] acetic acid as the purified enantiomer starting material. The optical rotation of the starting material was [α]25D=−0.4 (c=0.1, MeOH). 2-(methylsulfanyl)-10-{2-[(2S)-piperidin-2-yl] ethyl}-10H-phenothiazine dihydrochloride was produced in 95% yield by 1H NMR analysis and 95% by LCMS analysis and appeared as a green crystalline powder. The elemental analysis for 2-(methylsulfanyl)-10-{2-[(2S)-piperidin-2-yl] ethyl}-10Hphenothiazine dihydrochloride is C20H26Cl2N2S2, the formula weight is 429.4698, and the moisture content using the “Karl Fisher” method is 1.29% (by weight).

What is claimed is:

1. A composition comprising a purified enantiomer of a compound having the formula: 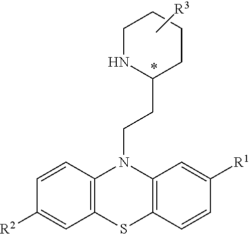
where R¹ is hydrogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, salt or a controlled release carrier.

2. The compound of claim 1 wherein R¹ is an alkylthio group, R² is hydrogen or a straight chain alkyl containing from one to about five carbon atoms, R³ is hydrogen, and the enantiomer is the (+) enantiomer.

3. The compound of claim 1 wherein R¹ is an alkylthio group, R² is hydrogen or a straight chain alkyl containing from one to about five carbon atoms, R³ is hydrogen, and the enantiomer is the (−) enantiomer.

4. The compound of claim 2 having the formula 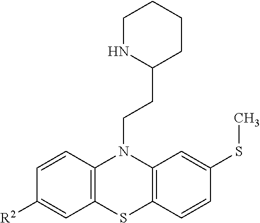
where R² is straight chain lower alkyl having from one to about three carbon atoms.

5. The compound of claim 3 having the formula 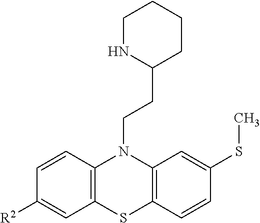
where R² is straight chain lower alkyl having from one to about three carbon atoms.

6. A method for treating a subject having a cancer in need of therapy thereof comprising administering to the subject a purified enantiomer of compound in an amount effective in inhibiting tumor growth, said compound having the formula: 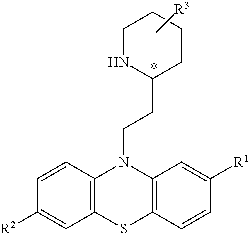
where R¹ is hydrogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, salt or a controlled release carrier.

7. The method of claim 5 wherein said compound is administered to a patient as monotherapy.

8. The method of claim 5 wherein said compound is administered to a patient receiving radiotherapy.

9. The method of claim 5 wherein said compound is administered to a patient receiving one or more chemotherapeutic or targeted cancer therapies, wherein said compound is administered before, during or after the chemotherapeutic or targeted therapy.

10. A method for treating a subject having a cancer and receiving immunotherapy comprising administering to the subject a purified enantiomer of a compound in an amount effective in inhibiting tumor growth, a purified enantiomer of said compound having the formula: 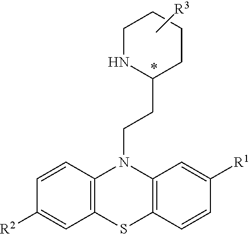
where R¹ is hydrogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, salt or a controlled release carrier.

11. The method of claim 9, wherein the immunotherapy comprises administering T-cells, NK killer cells or CART cells to said subject.

12. The method of claim 9, wherein the immunotherapy comprises administering a checkpoint inhibitor to said subject.

13. A pharmaceutical composition comprising a compound having the formula: 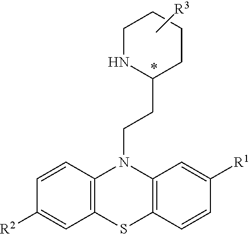
where R¹ is trifluoromethyl or an alkylthio group, R² is hydrogen, alkoxy or a branched or a straight chain acyclic alkyl group containing from three to about five carbon atoms, and R³ is hydrogen, lower alkyl or lower alkoxy, and a pharmaceutically acceptable carrier, salt or a controlled release carrier.

14. The pharmaceutical composition of claim 12 comprising a compound having the formula: 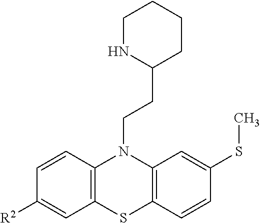
where R² is straight chain lower alkyl having three carbon atoms.

15. A composition comprising a purified enantiomer of a compound having the formula: 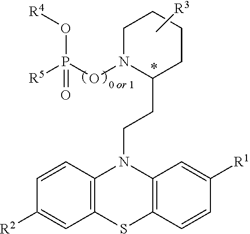
where R¹ is trifluoromethyl or an alkylthio group, R² is hydrogen, alkoxy or a branched or a straight chain acyclic alkyl group containing from three to about five carbon atoms, and R³ is hydrogen, lower alkyl or lower alkoxy, and a pharmaceutically acceptable carrier, salt or a controlled release carrier.

16. The composition of claim 14, where the compound has the formula:

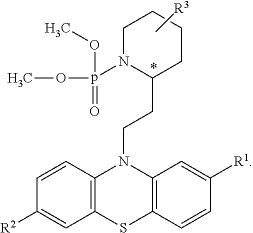

17. The composition of claim 14, where the compound has the formula: [chemical structure]
